(19) United States
(12) Patent Application Publication
(10) Pub. No.: US 2011/0269636 A1
(43) Pub. Date: Nov. 3, 2011

(54) MATERIALS AND METHODS FOR IDENTIFYING PATIENTS AT HEIGHTEN RISK FOR DEVELOPING HER2+ RELATED BRAIN TUMORS

(75) Inventors: Sunil Badve, Indianapolis, IN (US); Lang Li, Carmel, IN (US); George Sledge, Indianapolis, IN (US); Renata Duchnowaska, Warsaw (PL); Jacek Jassem, Gdansk (PL); Patricia Steeg, Laurel, MD (US)

(73) Assignee: INDIANA UNIVERSITY RESEARCH & TECHNOLOGY CORPORATION, Indianapolis, IN (US)

(21) Appl. No.: 12/955,217

(22) Filed: Nov. 29, 2010

Related U.S. Application Data
(63) Continuation of application No. PCT/US09/03330, filed on Jun. 1, 2009.
(60) Provisional application No. 61/057,828, filed on May 31, 2008.

(51) Int. Cl. C40B 30/04; G01N 33/574; C07K 16/22; C07K 16/32; C07K 16/40; C07K 16/18; C12Q 1/68; C07H 21/00

(52) U.S. Cl. 506/9; 435/6.11; 435/7.1; 536/24.31; 530/387.7; 530/389.7; 530/389.2

(57) ABSTRACT

Aspects of the invention include methods for identifying patients with HER2+ cancers that are at a heightened risk for developing brain metastasis within three years of having been diagnosed with HER2+ cancer. Some embodiments are methods that include the steps of contacting at least a portion of the tumor tissue from patients with probes that interact with the products of a set of thirteen genes that are expressed in these patients at markedly higher levels than in similarly situated patients that are not a heightened risk for developing brain metastasis within this three year window. In some embodiments the tissue samples are assayed from the presence of RNA indicative of the expression of member of a set of 13 genes identified as being differentially expressed in patients with and without a heightened risk for developing brain metastasis.

### PRIORITY CLAIM

This application is a continuation of PCT International Patent Application No. PCt/US09/03330, entitled “Materials and Method for Identifying Patients at Heightened Risk for Developing Her2+ Related Brain Tumors which claims the benefit under 35 U.S.A.C Section 119 of of U.S. provisional patent application No. 61/057,826 filed on May 31, 2008, the disclosures of which are expressly incorporated herein by reference.

### FIELD OF THE INVENTION

Various aspects relate to devices and methods that can be used to identify patients at a heightened risk for developing brain tumors thought to have metastasized from cancers originating in other organs including the breast.

### BACKGROUND

Brain metastasis is a serious complication of breast cancer and associated with extremely high morbidity and mortality. The survival of patients diagnosed with brain metastasis is extremely poor; less than 2% of women diagnosed with this disease are still alive two years after their diagnosis. Brain metastasis is particularly frequent in women whose tumors express the HER2 oncogene.

At the same time, the success of current HER2-directed therapies such as treatment with the drug trastuzumab has been associated with an almost 200% increase in the incidence of brain metastasis in patients with advanced breast cancer. Various hypotheses have been proffered to account for these observations including the direct promotion of brain metastases by HER-2 over-expression, poor penetration of the blood brain barrier by trastuzumab, and increased survival of patients with HER-2+ related cancers treated with new drugs such as trastuzumab.

Strategies for preventing and/or treating brain metastases of breast cancer are beginning to emerge. However, it not desirable to treat all patients at risk for developing HER-2+ metastatic diseases as though they have an equal propensity to develop this disease because many of these treatment regimes are associated with adverse side effects and the majority of patients with HER-2+ tumors are slow to develop this complication. Accordingly, there is a need to identify patients with an increased risk for developing metastases brain disease and to identify specific genes and gene products that can be targeted to treat these patients. One goal of the work present herein is to address these needs.

### SUMMARY OF THE INVENTION

Some aspects include a method of identifying patients having an elevated risk of developing brain metastasis, comprising the steps of: contacting a sample of tumor tissue with at least one probe that selectively binds under stringent conditions to an expression product of at least one gene selected from the group of genes consisting of: MYC, CDK4, CCNC, PTK2, BARD1, RAD 51, FANCG, PCNA, PRCC, TPR, EMS1, DSP, and HDGF; analyzing the sample in contact with the probes to identify which of the genes in the sample are expressed; and comparing the profile of the genes expressed with a panel of genes expressed in patients identified as being at high risk for developing brain metastasis within three years of diagnosis with HER2+ cancer.

In some embodiment the method of identifying patients further includes using a includes using at least one first probe that selectively binds to at least one gene product produced by expressing a gene selected from the first set of genes consisting of: CDK4, CCNC, PTK2 and MYC; at least one second probe that selectively binds under stringent conditions to at least one gene product produced by expressing at least one gene selected from the second group of genes consisting of: BARD1, RAD51 and FANCG under stringent conditions; and at least one third probe that selectively binds to at least one gene product produced by expressing at least one gene selected from the third group of genes consisting of PCNA, PRCC, TPR, EMS1, DSP and HDGF under stringent conditions. In some other embodiments that method for identifying patients with a heightened risk of developing brain metastasis within three years of diagnose includes contacted with probes that selectively binds under stringent conditions to gene produces produced by all of the following genes: MYC, CDK4, CCNC, PTK2, BARD1, RAD 51, FANCG, PCNA, PRCC, TPR, EMS1, DSP, and HDGF.

In some embodiments the tissue sample is processed by; extracting the total RNA from the primary tumor, to produce RNA which is reversed transcribed to form a set of cDNA molecules corresponding to the RNA in the tumor sample; amplifying the cDNA using quantitative polymerase chain reaction; and contacting the amplified cDNA with a set of probes that selectively bind under stringent conditions to the genes selected from the group consisting of: MYC, CDK4, CCNC, PTK2, BARD1, RAD 51, FANCG, PCNA, PRCC, TPR, EMS1, DSP, and HDGF. In some embodiments the probes are substantially affixed to a solid support.

In some aspects the method for identifying patients with a heightened risk of developing brain metastasis further including the step of: identifying tumors associated with a form of cancer that is likely to result in brain metastasis, within three years of first diagnosis, by detecting tumors that exhibit a statistically significant expression level of the genes selected from the group consisting of: MYC, CDK4, CCNC, PTK2, BARD1, RAD 51, FANCG, PCNA, PRCC, TPR, EMS1, DSP, and HDGF. In some embodiments the tumor samples are collected from patients that have undergone treatment with trastuzumab. In some embodiments the tumor samples are collected from patients that have been diagnosed with an HER2 related cancer.

Still another aspect of the invention is a kit for identifying patients with an increased risk for developing brain metastasis, comprising: at least one probe that selectively interacts under stringent conditions with a gene product produced by expressing at least one of the genes selected from the group of genes consisting of: MYC, CDK4, CCNC, PTK2, BARD1, RAD 51, FANCG, PCNA, PRCC, TPR, EMS1, DSP, and HDGF. Some embodiments are a kit which includes probes that selectively interact under stringent conditions with gene products produced by expressing all of the genes in the group consisting of; MYC, CDK4, CCNC, PTK2, BARD1, RAD 51, FANCG, PCNA, PRCC, TPR, EMS1, DSP, and HDGF. In some embodiments the probe(s) is substantially affixed to a solid support.

In some embodiments the kit for identifying patients with an increased risk for developing bran metastasis within three years of their initial diagnosis with cancer includes at least one probe that is a polynucleotide that is complimentary to a unique portion of DNA present in at least one of the genes selected from the group of genes consisting of MYC, CDK4, CCNC, PTK2, BARD1, RAD 51, FANCG, PCNA, PRCC, TPR, EMS1, DSP, and HDGF, and that binds to the unique portion of DNA under stringent conditions. Still other embodiments includes a set of polynucleotides that are complimentary to the unique portions of all of the genes selected from the group of genes consisting of: MYC, CDK4, CCNC, PTK2, BARD1, RAD 51, FANCG, PCNA, PRCC, TPR, EMS1, DSP, and HDGF and that bind to the unique portions of DNA under stringent conditions. In some embodiments the kit includes a device that can be used to measure the differential expression of the genes including a chip wherein the chip is suitable for DNA annealation selection and ligation.

In some embodiments the kit for identifying patients with an increased risk for developing bran metastasis within three years of their initial diagnosis with cancer includes a set of polynucleotide primers suited for use in quantitative real time PCR.

In still other embodiments the kit includes at least one probe that is a protein. The at least one protein probe preferentially binds to at least one protein or portion of at least one protein produced by the expression of at least one of the genes selected from the group of genes consisting of: MYC, CDK4, CCNC, PTK2, BARD1, RAD 51, FANCG, PCNA, PRCC, TPR, EMS1, DSP, and HDGF. In some embodiments the protein is an antibody. In another embodiment the kit includes a set of proteins that bind with high affinity to the set of proteins produced by expressing all of the genes selected from the group consisting of: MYC, CDK4, CCNC, PTK2, BARD1, RAD 51, FANCG, PCNA, PRCC, TPR, EMS1, DSP, and HDGF, in some of these embodiments the proteins are antibodies.

In some embodiments the kit for identifying patients with an increased risk for developing brain metastasis within three years of their initial diagnosis with cancer includes instructions for correlating the level of gene expression measured for the genes selected from the group consisting of MYC, CDK4, CCNC, PTK2, BARD1, RAD 51, FANCG, PCNA, PRCC, TPR, EMS1, DSP, and HDGF, with a patient's likelihood of developing brain metastasis within three years of the patent's original cancer diagnosis.

Development of a method for identifying patients with an increased risk in brain relapse in breast cancer patients diagnosed with HER2 positive tumors (HER-2+). Brain metastasis is a lethal complication of breast cancer and is particularly common in HER2+ patients; 80% of whom live for less than 1 year and 98% are dead by the 2^{nd} year. The success of HER2 targeted therapy (trastuzumab; Herceptin™) at most metastatic sites of the body has been marred by a 200% increase incidence of brain metastasis. Many of these patients had systemic disease that was controlled by trastuzumab, yet they succumbed to brain metastasis. Although the exact reasons for increased incidence of brain metastasis are not understood, poor penetration of blood-brain barrier by trastuzumab is a likely cause. Adjuvant trials of trastuzumab also show brain relapses, so earlier treatment does not solve this problem. We used DNA Annealation Selection and Ligation technology (DASL™, Illumina) to analyze gene expression on HER2+ primary tumors from a group of patients treated with trastuzumab and for which detailed follow-up data was available. We identified a 13-gene molecular signature that is predictive of the length of time to develop of brain metastasis. We have identified two molecular pathways, one enhancing the activity of HER2 itself (“super-HER2 phenotype”) in tumor cells and the other determining whether a cell can repair a serious DNA defect, double strand breaks, and thus survive chemotherapy. We hypothesize that these pathways are critical for the development of brain metastases. This gene signature will enable prospective identification of patients who are likely to develop brain metastases, and these patients will be candidates for screening and participation in trials testing potentially preventative therapies.

Some embodiments include methods of identifying patients that have an elevated risk of developing brain metastasis comprising the steps of: analyzing tissue sample from a patient, in which at least some cells in the tissue sample express at least one gene encoding at least one gene product selected from the group consisting of: (MYC), Cyclin Dependent Kinase 4 (CDK4), Protein Kinase 2 (PTK2), Cyclin C 1 (CCNC), Proliferating Cell Nuclear Antigen (PCNA), BRCA1 Associated Ring-Domain 1 (BARD), Fanconi Anemia Complementation Group G (FANCG), Rad51, Excess Microsporocytosis 1 (EMS1), Desmoplakin (DSP), Papillary Renal Cell Carcinoma (PRCC), the Met oncogene activator Translocated Promoter Region (TPR), Hepatoma Derived Growth Factor (HDGF), and the like; measuring the expression level of the gene; and comparing the expression level of the gene to a model for predicting brain metastasis, wherein the model correlates a change in the expression level of the gene with an increased risk of developing brain metastasis.

Other embodiments include methods of identifying patients that have an elevated risk of developing brain metastasis, comprising the steps of: obtaining a tissue sample from a patient diagnosed with a HER2+ related cancer; measuring the expression level in the tissue sample of at least one gene encoding a gene product selected from the group consisting of: Myc, Cyclin Dependent Kinase 4, Protein Kinase 2, Cyclin C 1, Proliferating Cell Nuclear Antigen , BRCA1 Associated Ring-Domain 1, Fanconi Anemia Complementation Group G, Rad51, Excess Microsporocytosis 1, Desmoplakin, Papillary Renal Cell Carcinoma, the Met oncogene activator Translocated Promoter Region, Hepatoma Derived Growth Factor, and the like; and comparing the measured expression levels of the gene with a model that correlates changes in the expression level of the gene with an increased risk for developing brain metastases within about 3 years of a diagnosis of cancer.

Still another embodiment is a method of identifying patients that have an elevated risk of developing brain metastasis, comprising the steps of: analyzing a tissue sample from a patient; measuring the expression level in the tissue sample of at least two to five genes encoding gene products selected from the group consisting of: Myc, Cyclin Dependent Kinase 4, Protein Kinase 2, Cyclin C 1, Proliferating Cell Nuclear Antigen, BRCA1 Associated Ring-Domain 1, Fanconi Anemia Complementation Group G, Rad51, Excess Microsporocytosis 1, Desmoplakin, Papillary Renal Cell Carcinoma, the Met oncogene activator Translocated Promoter Region, Hepatoma Derived Growth Factor, and the like; and comparing the measured expression levels of the at least two to five genes with a model that correlates changes in the expression level of the at least two to five genes with an increased risk for developing brain metastases within about 3 years of a diagnosis of cancer.

Yet another embodiment is a method of screening patients comprising the steps of: analyzing a tissue sample from a patient; measuring the expression level in the tissue sample of at least six to ten genes encoding gene products selected from the group consisting of: Myc, Cyclin Dependent Kinase 4, Protein Kinase 2, Cyclin C 1, Proliferating Cell Nuclear Antigen, BRCA1 Associated Ring-Domain 1, Fanconi Anemia Complementation Group G, Rad51, Excess Microsporocytosis 1, Desmoplakin, Papillary Renal Cell Carcinoma, the Met oncogene activator Translocated Promoter Region, Hepatoma Derived Growth Factor and the like; and comparing the measured expression levels of the at least six to ten genes with a computer generated model that correlates changes in the expression level of the at least two to five genes with an increased risk for developing brain metastases within about 3 years of a diagnosis of cancer.

One embodiment is a method of screening patients, comprising the steps of: analyzing a tissue sample from a patient diagnosed; measuring the expression level in the tissue sample of at least eleven to thirteen genes encoding gene products selected from the group consisting of: Myc, Cyclin Dependent Kinase 4, Protein Kinase 2, Cyclin C 1, Proliferating Cell Nuclear Antigen, BRCA1 Associated Ring-Domain 1, Fanconi Anemia Complementation Group G, Rad51, Excess Microsporocytosis 1, Desmoplakin, Papillary Renal Cell Carcinoma, the Met oncogene activator Translocated Promoter Region, Hepatoma Derived Growth Factor, and the like; and comparing the measured expression levels of the at least eleven to thirteen genes with a computer generated model that correlates changes in the expression level of the at least eleven to thirteen genes with an increased risk for developing brain metastases within about 3 years of a diagnosis of cancer.

Another embodiment is a method of identifying patients with an elevated risk of developing brain metastasis using at least one of the methods disclosed to test patients who have undergone treatment with trastuzumab.

Some other embodiments include a kit for identifying patients that have an increased risk for develop brain metastasis, comprising: a set of polynucleic acids that hybridize to at least a portion of at least one gene product selected from the group of gene products consisting of: Myc, Cyclin Dependent Kinase 4, Protein Kinase 2, Cyclin C 1, Proliferating Cell Nuclear Antigen, BRCA1 Associated Ring-Domain 1, Fanconi Anemia Complementation Group G, Rad51, Excess Microsporocytosis 1, Desmoplakin, Papillary Renal Cell Carcinoma, the Met oncogene activator Translocated Promoter Region, Hepatoma Derived Growth Factor, and the like; and a device or a reagent for measuring a change in the level of expression of the genes corresponding to at least one of the named genes. In one embodiment the set of polynucleic acids are attached to at least one surface. In one embodiment the device, composition or means used to measure the differential expression of the genes includes a chip wherein the chip is suitable for DNA Annealation Selection and Ligation. In still another embodiment the kit includes polynucleic acid primers suited for use in quantitative real time PCR and may optionally include buffers, enzymes and other components that can be used to perform the PCR.

Additional embodiments include a kit for identifying patients with an increased risk for developing brain metastasis, comprising: a set of proteins that bind to at least one protein selected from the group of proteins consisting of: Myc, Cyclin Dependent Kinase 4, Protein Kinase 2, Cyclin C 1, Proliferating Cell Nuclear Antigen, BRCA1 Associated Ring-Domain 1, Fanconi Anemia Complementation Group G, Rad51, Excess Microsporocytosis 1, Desmoplakin, Papillary Renal Cell Carcinoma, the Met oncogene activator Translocated Promoter Region, Hepatoma Derived Growth Factor, and the like; and at least one reagent for measuring a change in the level of expression of at least one of these proteins. In some embodiments the kits include at least one antibody that binds to a gene product whose differential expression correlates with an increased risk for developing brain metastasis.

Some embodiments are methods for identifying signature genes that can be used for predicting early brain metastasis in primary HER2+ breast tumor patients, comprising the steps of: analyzing at a least one tissue sample from a set of patients, wherein the set of patients include patients diagnosed with primary HER2+ breast cancer, or previously diagnosed with HER2+ breast cancer, a first subset of patients that develop brain metastasis and a second subset of patients that do not develop brain metastasis during the course of about 2-5 years after diagnosis, and measuring the level of expression of at least one gene in the tissue samples; monitoring the progress of at least some of the patients in the set of patients in order to identify patients that are in the first subset of patients and patients that are in the second subset of patients; and comparing differences in the expression level of the genes measured from patients in the first subset with patients in the second subset of patients and identifying a genetic signature indicative of an increased risk for develop brain metastasis

### BRIEF DESCRIPTION OF THE SEQUENCES

SEQ ID NO:1 provides the 5′ portion of a probe for CDK4 gene starting at position 837 of accession number NM_{—}0529.

SEQ ID NO:2 provides the 5′ portion of a probe for CDK4 gene starting at position 377 of accession number NM_{—}0529.

SEQ ID NO:3 provides the 5′ portion of a probe for CDK4 gene starting at position 781 of accession number NM_{—}0529.

SEQ ID NO:4 provides the 5′ portion of a probe for RAD51 gene starting at position 1088 of accession number NM_{—}0027.

SEQ ID NO:5 provides the 5′ portion of a probe for RAD51 gene starting at position 1387 of accession number NM_{—}0027.

SEQ ID NO:6 provides the 5′ portion of a probe for RAD51 gene starting at position 1542 of accession number NM_{—}0087.

SEQ ID NO:7 provides the 5′ portion of a probe for PRCC gene starting at position 760 of accession number NM_{—}00597.

SEQ ID NO:8 provides the 5′ portion of a probe for PRCC gene starting at position 988 of accession number NM_{—}00597.

SEQ ID NO:9 provides the 5′ portion of a probe for PRCC gene starting at position 1223 of accession number NM_{—}00597.

SEQ ID NO:10 provides the 5′ portion of a probe for EMS1 gene starting at position 755 of accession number NM_{—}13856.

SEQ ID NO:11 provides the 5′ portion of a probe for EMS1 gene starting at position 2014 of accession number NM_{—}13856.

SEQ ID NO:12 provides the 5′ portion of a probe for EMS1 gene starting at position 1739 of accession number NM_{—}13856.

SEQ ID NO:13 provides the 5′ portion of a probe for PTK2 gene starting at position 3719 of accession number NM_{—}15383.

SEQ ID NO:14 provides the 5′ portion of a probe for PTK2 gene starting at position 424 of accession number NM_{—}15383.

SEQ ID NO:15 provides the 5′ portion of a probe for PTK2 gene starting at position 3433 of accession number NM_{—}15383.

SEQ ID NO:16 provides the 5′ portion of a probe for MYC gene starting at position 1630 of accession number NM_{—}00246.

SEQ ID NO:17 provides the 5′ portion of a probe for MYC gene starting at position 1527 of accession number NM_{—}00246.

SEQ ID NO:18 provides the 5′ portion of a probe for MYC gene starting at position 1344 of accession number NM_{—}00246.

SEQ ID NO:19 provides the 5′ portion of a probe for PCNA gene starting at position 315 of accession number NM_{—}18264.

SEQ ID NO:20 provides the 5′ portion of a probe for PCNA gene starting at position 122 of accession number NM_{—}18264.

SEQ ID NO:21 provides the 5′ portion of a probe for PCNA gene starting at position 953 of accession number NM_{—}18264.

SEQ ID NO:22 provides the 5′ portion of a probe for TPR gene starting at position 3951 of accession number NM_{—}00329.

SEQ ID NO:23 provides the 5′ portion of a probe for TPR gene starting at position 5294 of accession number NM_{—}00329.

SEQ ID NO:24 provides the 5′ portion of a probe for TPR gene starting at position 3443 of accession number NM_{—}00329.

SEQ ID NO:25 provides the 5′ portion of a probe for BARDI gene starting at position 1654 of accession number NM_{—}00046.

SEQ ID NO:26 provides the 5′ portion of a probe for BARDI gene starting at position 509 of accession number NM_{—}00046.

SEQ ID NO:27 provides the 5′ portion of a probe for BARDI gene starting at position 2027 of accession number NM_{—}00046.

SEQ ID NO:28 provides the 5′ portion of a probe for DSP gene starting at position 2858 of accession number NM_{—}00441.

SEQ ID NO:29 provides the 5′ portion of a probe for DSP gene starting at position 3175 of accession number NM_{—}00441.

SEQ ID NO:30 provides the 5′ portion of a probe for DSP gene starting at position 2592 of accession number NM_{—}00441.

SEQ ID NO:31 provides the 5′ portion of a probe for HDGF gene starting at position 1603 of accession number NM_{—}00449.

SEQ ID NO:32 provides the 5′ portion of a probe for HDGF gene starting at position 740 of accession number NM_{—}00449.

SEQ ID NO:33 provides the 5′ portion of a probe for HDGF gene starting at position 1258 of accession number NM_{—}00449.

SEQ ID NO:34 provides the 5′ portion of a probe for FANCG gene starting at position 2489 of accession number NM_{—}00462.

SEQ ID NO:35 provides the 5′ portion of a probe for FANCG gene starting at position 1440 of accession number NM_{—}00462.

SEQ ID NO:36 provides the 5′ portion of a probe for FANCG gene starting at position 1004 of accession number NM_{—}00462.

SEQ ID NO:37 provides the 5′ portion of a probe for CCNC gene starting at position 891 of accession number NM_{—}00519.

SEQ ID NO:38 provides the 5′ portion of a probe for CCNC gene starting at position 567 of accession number NM_{—}00519.

SEQ ID NO:39 provides the 5′ portion of a probe for CCNC gene starting at position 708 of accession number NM_{—}00519.

### BRIEF DESCRIPTION OF THE FIGURES

Aspects and various embodiments of the disclosure and the manner of obtaining them will become more apparent and will be better understood by reference to the following description of the embodiments taken in conjunction with the accompanying figures, charts, drawing, schematics, formula, sequences and the like, as follows.

FIG. 1a Graph illustrating the probability of brain relapse free survival plotted against number of days diagnosed using the 13-gene signature disclosed herein. Data generated using a training set, (n=60, event=31) with a P value of <0.0001.

FIG. 1b Graph illustrating the probability of brain relapse free survival plotted against number of days diagnosed using the 13-gene signature disclosed herein. Data generated for a validation set, (n=30, event=12) with a P value of <0.0ss and an HR value of 5.26.

FIG. 2 Results of quantitative real time PCR (qRT-PCR) analysis of 10 patients; CDK4 expression was measured to confirm the data obtained by DASL analysis.

FIG. 3 A heat map illustrating results of a Yes/No analysis of differences in brain metastasis relapse between patients with HER2 tumors.

FIG. 4 A heat map illustrating results obtained by measuring the differential expression of genes between patients with early brain metastasis (≦3 years) and patients with a late development of brain metastasis (≧3 years).

FIG. 5 Table 1. Summary of various genes identified in the thirteen gene signature whose expression correlates well with the development of brain metastasis within three years of diagnosis with breast cancer.

FIG. 6 Table 2. Summary of the clinical traits of patients whose tumor samples and outcomes were used in a study to create and validate various models for predicting brain metastasis.

FIG. 7 Table 3. Summary of individual variable contributions of various biomarkers to the prediction of brain metastasis complied with an n value of 90.

FIG. 8 Table 4. A summary of some of the polynucleotide probes that specific for of a 13 gene signature found in HER2 cancer patients disclosed herein.

### DETAILED DESCRIPTION

The embodiments of the present invention described below are not intended to be exhaustive or to limit enabled and described aspects and embodiments of the disclosure to the precise forms disclosed in the following detailed description. Rather, the descriptions, examples and demonstrative experiments are chosen and described so that others skilled in the art may appreciate and understand the principles and practices of various aspects and embodiments of the disclosure.

Advances in chemotherapy and radiotherapy techniques have significantly improved loco-regional therapy for breast cancer. However, these improvements underscore the lack of advances in treatment of distant disease, particularly at niche sites. Brain metastasis, for instance, historically occurred in 10-15% of patients with metastatic disease. These patients have a poor prognosis with 80% of patients being dead within one 1 year of their initial diagnosis, due to both systemic and CNS disease.

Several recent studies have shown that patients, whose tumors are HER2+ (3+ or amplified) have a greater propensity to develop brain metastasis. In contrast to older studies, many of these patients have systemic disease that is either stable or responding to treatment, and up to 50% die of their CNS disease. Although the exact reasons for this increased incidence of CNS metastasis are not understood, one hypothesis proffered to account for this phenomenon is the proposed poor penetration of blood-brain barrier by drugs such as trastuzumab (Herceptin™). The average risk of developing of CNS metastasis in metastatic patients receiving trastuzumab is in the range of about 25-48%. Our group analyzed gene expression using DNA Annealation Selection and Ligation technology (DASL™, Illumina) in a cohort of HER2+ primary tumors recovered from women with breast cancer. In this study, 80% of these women had received trastuzumab as a part of their therapy for metastatic disease. Most of the women in this study were the subject of detailed follow-up, including registering how many of them went on to develop brain metastasis during the course of the study. During the course of this study, we identified a 13-gene molecular signature that is predictive of a reduced time interval between diagnosis with HER-2+ breast cancer and the development of brain metastasis relative to patients that did not exhibit differential expression of these 13 genes.

Historically, brain metastasis (Br-Met), occurred in about 10-15% of patients with metastatic breast cancer (MBC) and are associated with an extremely poor prognosis; the median survival term 3-6 months while, 2-year survival rates are under 2%. Patients with HER2+ tumors (3+ or FISH amplified) have a greater propensity to develop Br-Met metastasis. For example, a recent study reported that about 44% of 126 breast cancer patients with Br-Mets were HER2+.

According to the medical literature the incidence of Br-Met in HER2+ patients with MBC is increasing. By way of explanation and not limitation and without being bound by any theory a brief discussion of possible reasons for this development is as follows. Various theories have been proffered to the observed increase in Br-Met since the introduction of trastuzumab (Herceptin™) therapy. increase have been proffered. For example, some researchers have partially ascribed the increase in patients with Br-Met to the poor penetration of the blood-brain barrier by drugs such as trastuzumab. The ratio of the levels of trastuzumab in cerebrospinal fluid to the levels of trastuzumab in the blood is about 1:300-400; and a mild increase in the levels following whole brain radiation has also been reported. In a retrospective study of 122 patients treated with trastuzumab—alone or in combination with chemotherapy, for example, Bendell. et al, found significantly higher incidence of Br-Met (34%) (in patients treated with trastuzumab) with almost half of all patients dying due to progressive CNS disease. See, Bendel,l J. C. et al, “Central Nervous System Metastases in Women Who Received Trastuzumab-Based Therapy for Metastatic Breast Carcinoma Cancer,” 97. 2972-2977 (2003). Similarly, Clayton et al. reported a high rate of brain relapse in patients treated with trastuzumab for MBC, despite this therapy exhibiting effective disease control at extra-cranial sites. See, Clayton, A. J., et al., “Incidence of Cerebral Metastases in Patients Treated with Trastuzumab for Metastatic Breast Cancer,” Br J Cancer, 91, 639-643 (2004). At progression, CNS lesions were the first site of symptomatic disease in 82% of cases. The average risk of development of CNS metastasis appears to be doubled in patients receiving trastuzumab. Without being bound by any therapy or explanation some researchers have partially attributed this to better control of extra-cranial disease and the increased survival associated with trastuzumab therapy. Accordingly, preventing non-CNS metastasis, the use of adjuvant trastuzumab may lead to a relative increase in Br-Met amongst patients with HER2+ MBC.

One approach disclosed herein to address the problem of Br-Mets was to identify a gene expression signature in HER2+ primary tumors that predicts the time of the development of Br-Met, and then to examine the genes to determine if they are good therapeutic targets. Once high-risk patients are identified, they may be offered prophylactic therapies such as whole brain radiation—a therapy too toxic for non-selective use in otherwise asymptomatic patients. Similarly, patients with this gene signature entered onto clinical trials for the validation of less toxic preventative strategies.

As described herein through a detailed analysis of gene expression patterns between HER2 patients that develop Br-Met with three of their initial diagnosis with HER2 cancers and those that do not we identified a 13 gene signature that is predictive for the identification of patients that develop brain metastasis with three years versus patents that are free of Br-Met for at least three years after their initial diagnosis of cancer. Patients that express the following thirteen genes MYC, CDK4, CCNC, PTK2, BARD1, RAD 51, FANCG, PCNA, PRCC, TPR, EMS1, DSP, and HDGF are at a statistically significant greater risk for early onset brain metastasis then similarly situated patients that do not. Interesting, while some of these genes have reportedly been associated with HER2 cancers many have not.

Studies conducted by Duchnowska, et al. (Co-I) in Poland, used a multi-institutional cohort of 264 cases of HER2+ breast cancers with Br-Met and analyzed these patients for parameters associated with the development of Br-Mets. See, Duchnowska, R., et al., “Brain Metastasis in HER-2 Positive Metastatic Breast Cancer (MBC) patents,” Eur J. Cancer, 165-166 (2006). This study found a significant association between pre-menopausal status, high histologic grade and early distant relapse of cancer (<2 years) and Br-Met.

### HER2 Related Genes

Several of the genes in the 13-gene signature have been implicated in cancer progression and have been linked to HER2 prognosis or biology. At least two of these genes are thought to be linked in the same molecular pathway. MYC expression in HER2+ breast cancer patients is associated with poor prognosis, but these patients show a significant benefit when they were treated with trastuzumab in the NSABP-31 clinical trial. Also, Cyclin dependent Kinase 4 (CDK4) is thought to play an important role in the control of G1/S transition of mitotic cell cycle.

In Estrogen Receptor (ER) Tumors, a Fold Increase in CDK4 Expression was Associated with 3 Times More Risk in Br-Met in Our Analysis (p=0.05).

CDK4 expression in breast cancer has been associated with large tumors, positive nodal status and recurrence. CDK4 knockout mice are resistant to HER2 tumorigenesis. And trastuzumab reportedly inhibits the expression level of cyclin D and c-MYC, thereby contributing to the release of the cell cycle inhibitor p27 from cyclin D:CDK4/6 complexes and increasing its effect on the inhibition of CDK2:cyclinE complexes. This finding links two of the 13 genes, CDK4 and MYC; and Protein tyrosine kinase 2 (PTK2), also called Focal adhesion kinase (FAK), is thought to be involved in integrin-mediated signaling pathway; expression has been correlated with shorter survival and with HER2+ status in node-negative breast cancer. Signaling through FAK is thought to be critical for HER2/HER3 cooperation in both tumor cell transformation and motility.

### Proliferation Related Genes

In addition to MYC and CDK4, at least two additional genes may be involved in proliferation. These include: Cyclin C (CCNC), which is a secondary cyclin associated with the G1 phase of the cell cycle, amplification of this gene is frequently seen in breast cancer. Although the role of cyclins in breast cancer has been well documented, cyclin C remains poorly characterized. Proliferating cell nuclear antigen (PCNA), which is a nuclear protein associated with delta-DNA polymerase cofactor complex, is commonly used as a measure of proliferation. It is also thought to play a role in regulation of DNA replication.

### DNA Repair Associated Proteins

The 13-gene signature contains disclosed herein includes a surprising large number of genes that encode proteins which have been characterized as DNA repair proteins. Perhaps more surprisingly, at least 3 of these proteins function in a single pathway, mediating the repair of DNA double strand breaks (DSBs), this is one of the most difficult DNA lesions to repair as it may require homologous recombination. These DNA repair proteins include the following: BRCA1 associated ring-domain 1 (BARD1) is thought to be the predominant binding partner of BRCA1. Binding is activated by detection of DNA DSBs and confers a potent E3 ubiquitin ligase activity. In DSB repair, the BARD1-BRCA1 interaction is thought to influence the location and stability of the Ub-FANCD2 complex. In addition, BARD1 can also induce p53-independent apoptosis in response to genotoxic stress and modulate mitotic spindle assembly. Expression, both in the nucleus and cytoplasm, has been documented; the latter being associated with poor prognosis in breast cancer. Fanconi Anemia,_complementation group G (FANCG) is a part of the Fanconi anemia core complex that mono-ubiquitinates FANCD2/FANCI to trigger recruitment of the repair complex to the DNA DSB. In this complex, FANCG binds BRCA2 and is thought to coordinate the release of RAD51 onto single stranded DNA in homologous recombination. FANCG is also a tumor suppressor, and Rad51 recombinase binds single stranded DNA at the edge of the DSB to form a helical nucleoprotein filament that then invades and pairs with homologous sequences in duplex DNA, initiating the strand-exchange reactions that underlie homologous recombination. It has been associated with poor prognosis in breast cancer. In addition, PCNA also participates in DNA repair.

Excess Microsporocytosis 1 (EMS1; also known as Cortactin) is thought to play a role in actin assembly; it appears to be required for the formation of functional invadopodia in tumor cell migration. This protein also appears to play a role in CD44 mediated invasion, ligand-induced down regulation of the Epidermal growth factor receptor (EGFR), and adhesion of breast cancer cells to bone marrow endothelial cells. Its expression has been associated with a poor prognosis in ER-negative but not ER+ breast cancers. Desmoplakin (DSP), a cytoskeletal protein, is associated with epidermal differentiation and control of cell shape and size. Papillary renal cell carcinoma (PRCC) gene function is not known. Translocated promoter region (to activated Met oncogene) (TPR) is a nuclear pore protein thought to play a role in controlling nucleo-cytoplasmic transport. And lastly, hepatoma derived growth factor (HDGF), is a gene whose function in breast cancer prognosis is poorly understood.

Genes identified in this survey include both genes that were thought to be associated with HER2+ cancers which was unexpected as these patients had already been treated with a drug known to effectively address these types of tumors. This group of genes also includes genes that are involved in DNA repair, another unexpected development as these genes were not necessarily thought to be associated with this particular form of cancer or in the selection of cancer cells that are especially suitable for brain metastasis.

### EXAMPLES

### Materials and Methods

Conditions constituting stringent hybridization conditions between oligonucleotides including probes and targets may vary with the length and composition of the hybridizing pair. General parameters for determining these conditions can be found, for example, in Sambrook, et al., Molecular Cloning-A laboratory Manual (2^{nd} Ed.) Vols. 1-3 Cold Spring Harbor laboratory, Cold Spring, N.Y. (1989) and similar texts.

Methods for preparing total ply(A)+ RNA are well known in the literature and described in text such as, “Current Protocols in Molecular Biology,” vol. 2, Current Protocols Publishing New York (1994) and Sambrook, et al., Molecular Cloning-A laboratory Manual (2^{nd} Ed.) Vols. 1-3 Cold Spring Harbor laboratory, Cold Spring, N.Y. (1989).

Some embodiments use or comprise the construction of a microarrays of specific polynucleotide microarrays. These microarrays may be constructed to measure the various parameters including, for example, the level of cDNA in a given sample. Commonly, microarrays are constructed by selecting at least one polynucleotide sequence that hybridizes to a marker of interest under the desired hybridization conditions. Probes may comprise DNA sequences, RNA sequences or copolymer sequences of both DNA and RNA the sequences can be made in vivo, in vitro using either biological systems, chemical or enzymatic methods or a hybrid of any or all of these approaches. The probes themselves may be comprised of naturally occurring or synthetic nucleotides.

The probes may be immobilized to a solid surface such as a nitrocellulose or nylon surface, filter material, or other surface by either the 5′ or 3′ end. Such hybridization processes are known and can be reviewed in, for example, Sambrook, et al., Molecular Cloning-A laboratory Manual (2^{nd} Ed.) Vols. 1-3 Cold Spring Harbor laboratory, Cold Spring, N.Y. (1989).

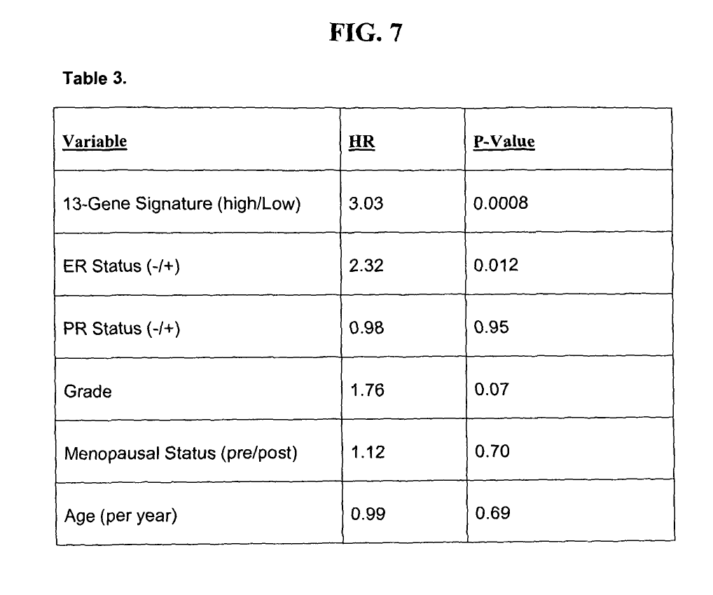

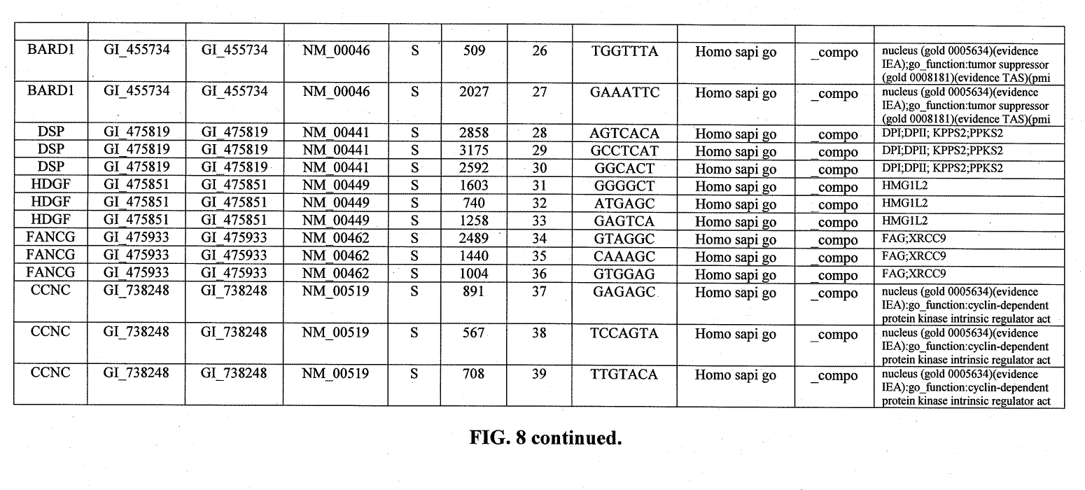

Sample of tumor tissue for a number of patients was analyzed to measure the expression of ˜500 cancer-associated genes. These sample were collected from patients in an on going study of individual diagnosed with HER2 type cancers. The clinical characteristics of the typical patient in this study is presented in Table 2 (FIG. 6). The sample used in the assay were processed using techniques well know in art tissue samples formalin fixed, paraffin embedded (FFPE) blocks of HER2+ primary tumors (n=90) using modified RT-PCR technology available through the Translational Genomics Core at IU Simon Cancer Center (PI is the Director). This DASL (DNA Annealation Selection and Ligation) technology permits high-throughput analysis and is optimally suited for analysis of FFPE samples. DASL assay was performed using the Sentrix Universal Array (Illumina Corp., San Diego, Calif.) of 502 known cancer genes. These assays were conducted as per the manufacturer's instructions. These assays included polynucleotide probes that bind under stringent conditions to specific genes. A summary of the types of polynucleotide probes that can be used to practice some embodiments of the invention are summarized in FIG. 8.

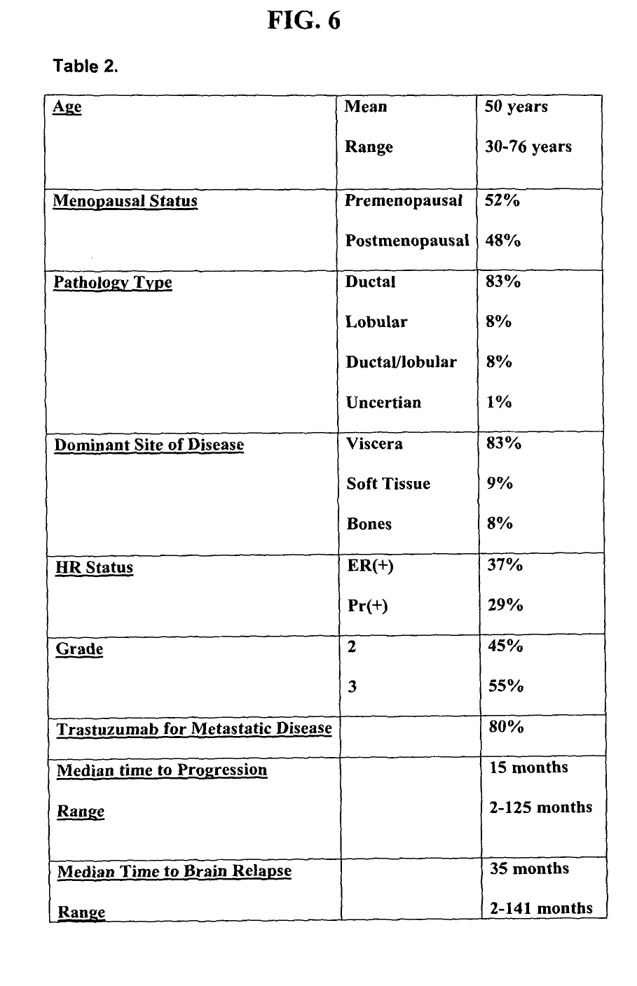

A DASL Illumina gene expression panel is normalized by their medians. Then, the gene expression data was log-transformed. Data analysis is performed via predictive analysis of microarray (PAM). The outcome variable was the time from diagnosis to brain metastasis. A set of 60 samples were used for training, and the other 30 samples were used for internal validation. The results were analyzed in accordance with the approach outline by Tibshirani, Hastie, Narasimhan and Chu, in “Diagnosis of multiple cancer types by shrunken centroids of gene expression”, PNAS 2002 99:6567-6572. These analysis demonstrated that 13 genes of the 502 assayed were differentially expressed in tumor cells taken from patients with a heightened risk for developing Br-MET these 13 genes are summarized in Table 1 (FIG. 5).

Three separate probe sets (that resemble RT-PCR) per gene are used to overcome RNA degradation. The data obtained using this approach is consistent and reproducible, i.e., the within-chip between-probe coefficient variance is around 4%, and the between-chip technical duplication coefficient variance is around 7%. Of the 90 patients analyzed, about, 80% had received trastuzumab as a part of their therapy. Forty-six patients were postmenopausal; there were 55 ER− and 33 ER+; 62 PR− and 26 PR+ patients. The frequencies of grades 1, 2, 3 are 1, 36, 46, respectively, there were incomplete data sets for seven of patients. Time to progression (TTP) ranged from 2-125 months (median 15 months); time to Br-Met ranged from 2-141 months (median 35 months). The DASL Illumina gene expression data after normalization by the cohort's medians was log-transformed. Data analysis was performed through significant analysis of microarray (SAM) and predictive analysis of microarray (PAM). The outcome variables we used were: 1) Br-Met-Yes vs. No; and 2) time from diagnosis to Br-Met. A random set of 60 samples are used for training the model, and the other 30 for internal validation of the model. This analysis revealed a molecular signature of Br-Met that is relevant to HER2+ MBC patients, and it can be used to propose additional studies in order to develop more effective therapies for the diagnosis and treatment of Br-Met.

### Experiment 1

### Brain Metastasis—Yes Versus No Analysis

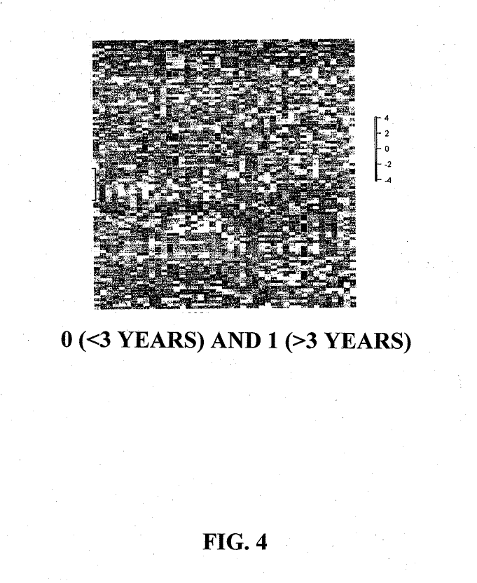

Referring now to FIG. 3. Specific genes followed on the in the study are plotted on the y-axis as a function of data collected from individuals tumor samples. Forty-three of the patients experienced brain metastasis within the time frame of the study and forty-seven did not. Of the 502 genes analyzed in this assay 25 genes were differentially expressed between the two groups having p value of <0.05, and a false discovery rate equal to 1. Light shades indicate genes that are over-expressed relative to the norm while dark shades indicate genes that are under-expressed relative to the norm.

Gene expression in the primary tumors of 43 patients with Br-Mets were compared to gene expression in patients without Br-Mets. Among 502 genes assayed, 25 genes with a p-value<0.05 were identified (false discovery rate (FDR)=1.00). This indicates that all of the “significant” genes could have been identified solely by chance. Accordingly, there appears to be is no solid evidence that HER2+ tumors have different gene expression patterns in patients with Br-Met than in patients without Br-Met. These data suggest that the development of Br-Met in patients with advanced HER2+ breast cancer is a stochastic event and that each and every patient, and that if she survives long enough, she could eventually develop Br-Mets.

### Experiment 2

Time from Diagnosis to Development of Brain Metastasis (Early Versus Late)

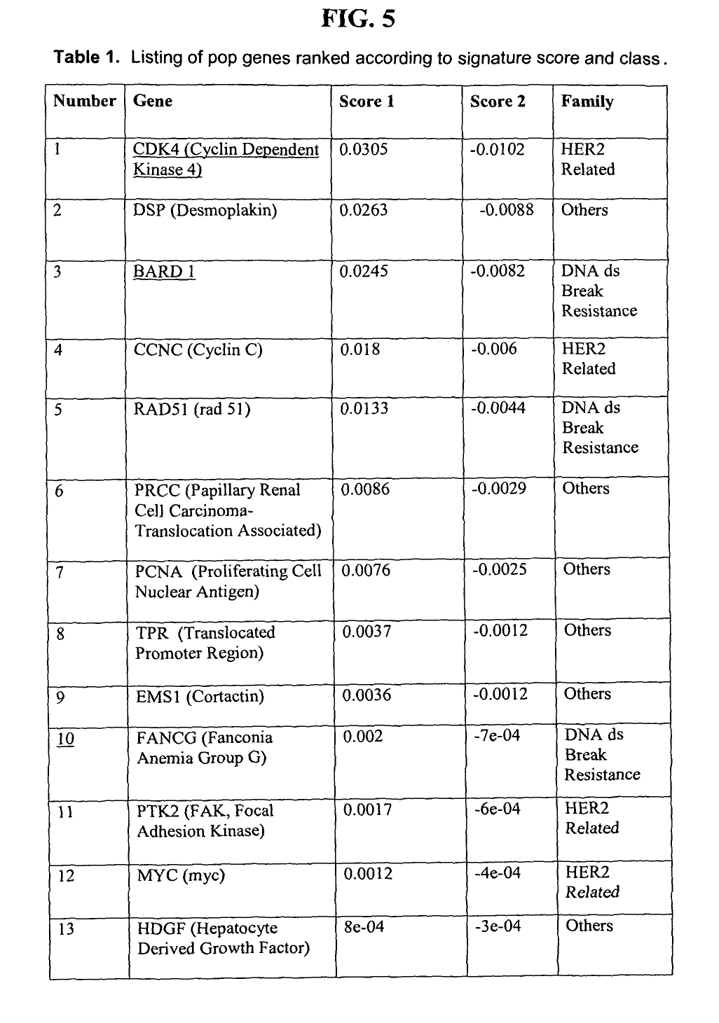

Referring now to FIG. 4. A heat map generated by measuring the differential expression of genes between patients with early brain metastasis (≦3 years) and patients with a late development of brain metastasis (≧3 years); 22 patients with brain metastasis within three years of their initial diagnosis were compared with 21 patients without brain metastasis after three years (medial of 51 months). Specific genes followed on the in the study are plotted on the y-axis as a function of data collected from individuals. Out of 502 genes analyzed 95 were found to be differentially expressed between the two groups the p value was <0.05, with a false discovery rate equal to 0.25. And 48 genes were differentially expressed with a p value of <0.01 and a false discovery rate equal to 0.1. Light shades indicate genes that are over-expressed relative to the norm while dark shades indicate genes that are under-expressed relative to the norm.

This analysis compared 22 patients with HER2+ tumors who developed Br-Mets within three years (median 22 months) from the time of their diagnosis (“early”) to 21 patients who developed Br-Met more than three years (median 51 months) after diagnosis (“late”). Another 47 censored patient samples were excluded from this analysis because we could not accurately determine their metastasis dates. Among the 502 genes assayed, 95 genes were differentially expressed among “early” and “late” Br-Met patients (FDR=0.1). Accordingly, the top 48 genes could include both reliable molecular targets and prognostic markers. In order to combine information from both metastasis patient samples and patient samples with censored metastasis time, a Cox proportional hazard model was fitted to the gene expression data. In order to perform this analysis, the patient set was sub-divided into sets and validation sets and a gene-signature was developed. The most prognostically significant genes identified from this data are, a 13-gene signature that is predictive of the time required to develop Br-Met (disease free survival, DFS).

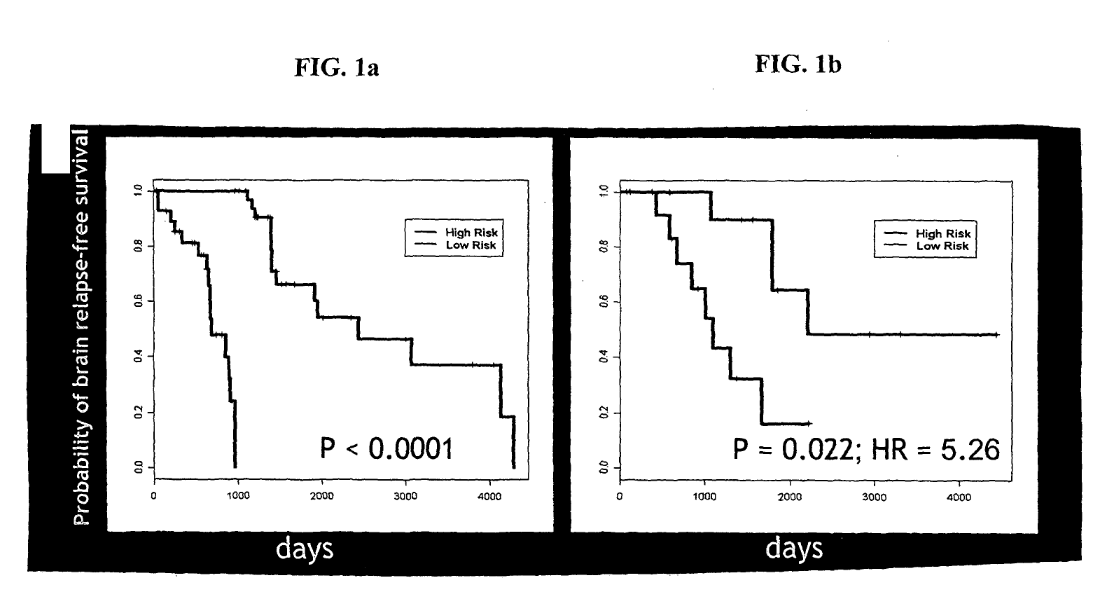

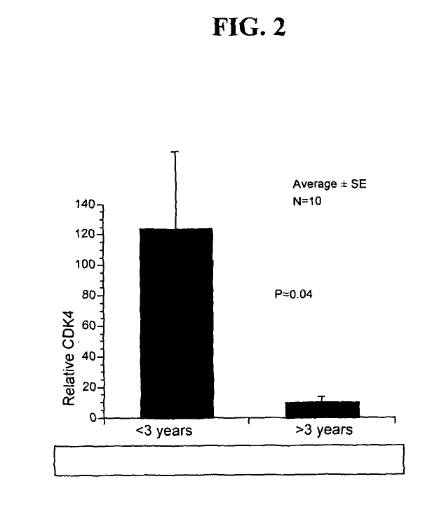

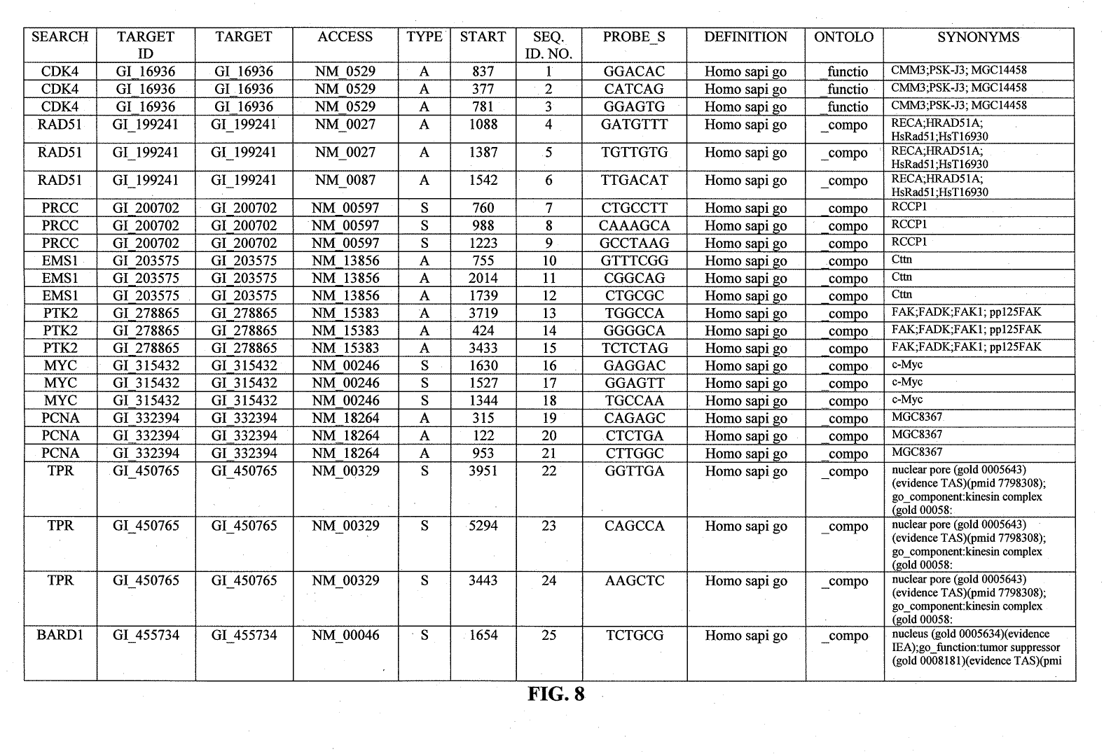

Referring now to FIGS. 1a and 1b. A similar analysis of the patients tracking the expression of the thirteen gene signature on the probability of brain relapse free survival also demonstrated a significant correlation between the expression of these genes and this parameter. Referring now to FIG. 7 (Table 3) summarized herein are the individual contributions to variable contributions to models predictive of the propensity for individuals to develop Br-Met within about 3 years of their initial diagnosis with cancer. As illustrated by the parameters summarized in Table 3 the 13 gene signature disclosed herein exhibited the greatest predictive power of all of the variable followed in this study of individual tumor samples.

### Experiment 3

### Confirmation of the DASL Data by RT-PCR Analysis

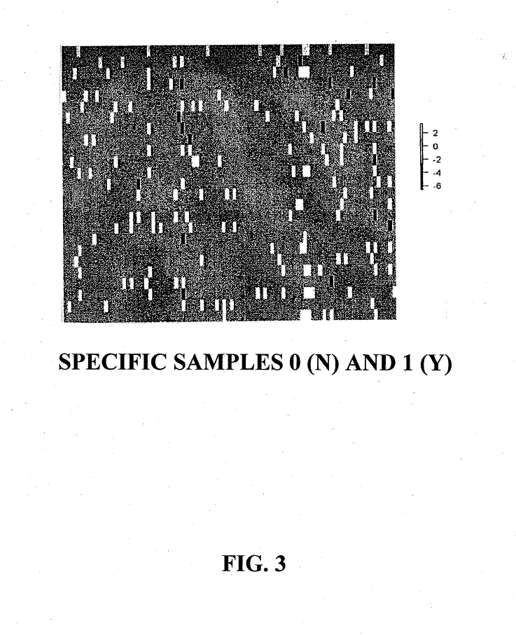

We performed confirmatory analysis of the DASL expression data using quantitative RT-PCR (qRT-PCR) on 3 of the genes included in the 13 gene signature discussed above. These analyses confirm the existence of similar differences in expression of the gene observed in the DASL analysis (e.g., see the data collected for CDK4 illustrated in FIG. 2).

### Experiment 4

After reviewing H&E slides, 3 10μ thick sections are obtained from the paraffin block (one block per case) on non-charged glass slides taking due precautions to avoid contamination. Macro-dissection is performed as required. Sections after de-paraffinization (CitriSolv®, Fisher Scientific, Fair Lawn, N.J.) is scraped into a micro-centrifuge tube. RNA is extracted using High Pure RNA Paraffin Kit (Roche Applied Bioscience, Indianapolis, Ind.). RNA from cases containing 200 ng/5 μl is pre-qualified using iScript (Bio-Rad Laboratories Inc., Hercules, Calif.) to reverse transcribe and SYBR Green Master Mix (Applied Biosystems, Foster City, Calif.) to perform quantitative PCR for the house-keeping gene (RPL13a). DASL assay is performed using the Sentrix Universal Array (Illumina Corp., San Diego, Calif.) of 502 known cancer genes as per the manufacturer's instructions. Technical duplicates are also included. For cases that yield less than 200 ng/5 μl total RNA, RNA amplification is performed using the WT-Ovation™ FFPE system. This system enables true amplification from samples with very low yields (50 ng of total RNA) with high r² values (FIG. 4).

RNA from cases that do not meet the RNA requirement for DASL, even after amplification, will be analyzed for the expression of only 13 genes, using SYBR Green Master Mix. Expression of GAPDH, RPLPO, beta-actin (as in Oncotype Dx) is used as a control to normalize the data. Some polynucleotide probes that can be used to assay for the 13 genes of interest are listed, for example, in FIG. 8 Table 4.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

We claim:

1. A method of identifying patients having an elevated risk of developing brain metastasis, comprising the steps of:
contacting a sample of tumor tissue with at least one probe that selectively binds under stringent conditions to an expression product of at least one gene selected from the group of genes consisting of: MYC, CDK4, CCNC, PTK2, BARD1, RAD 51, FANCG, PCNA, PRCC, TPR, EMS1, DSP, and HDGF;
analyzing the sample in contact with the probes to identify which of the genes in the sample are expressed; and
comparing the profile of the genes expressed with a panel of genes expressed in patients identified as being at high risk for developing brain metastasis within three years of diagnosis with HER2+ cancer.

2. The method according to claim 1, wherein the at least one probe includes:
at least one first probe that binds under stringent conditions to at least one gene product produced by expressing a gene selected from the first set of genes consisting of: CDK4, CCNC, PTK2 and MYC;
at least one second probe that binds under stringent conditions to at least one gene product produced by expressing at least one gene selected from the second group of genes consisting of: BARD1, RAD51 and FANCG; and
at least one third probe that binds under stringent conditions to at least one gene product produced by expressing at least one gene selected from the third group of genes consisting of PCNA, PRCC, TPR, EMS1, DSP and HDGF.

3. The method according to claim 1, wherein the tissue sample is contacted with probes to gene produces produced by all of the following genes: MYC, CDK4, CCNC, PTK2, BARD1, RAD 51, FANCG, PCNA, PRCC, TPR, EMS1, DSP, and HDGF.

4. The method according to claim 1, wherein the tissue sample is processed by;
extracting the total RNA from the primary tumor, to produce RNA which is reversed transcribed to form a set of cDNA molecules corresponding to the RNA in the tumor sample;
amplifying the cDNA using quantitative polymerase chain reaction; and
contacting the amplified cDNA with a set of probes that selectively bind under stringent conditions to the genes selected from the group consisting of: MYC, CDK4, CCNC, PTK2, BARD1, RAD 51, FANCG, PCNA, PRCC, TPR, EMS1, DSP, and HDGF.

5. The method according to claim 4, wherein the probes are substantially affixed to a solid support.

6. The method according to claim 1, further including the step of:
identifying tumors associated with a form of cancer that is likely to result in brain metastasis, within three years of first diagnosis, by detecting tumors that exhibit a statistically significant expression level of the genes selected from the group consisting of: MYC, CDK4, CCNC, PTK2, BARD1, RAD 51, FANCG, PCNA, PRCC, TPR, EMS1, DSP, and HDGF.

7. The method according to claim 1, wherein the tumor samples are collected from patients that have undergone treatment with trastuzumab.

8. The method according to claim 1, where in the tumor samples are collected from patients that have been diagnosed with an HER2 related cancer.

9. A kit for identifying patients with an increased risk for developing brain metastasis, comprising:
at least one probe that selectively interacts under stringent conditions with a gene product produced by expressing at least one of the genes selected from the group of genes consisting of: MYC, CDK4, CCNC, PTK2, BARD1, RAD 51, FANCG, PCNA, PRCC, TPR, EMS1, DSP, and HDGF.

10. The kit according to claim 9, further including probes that selectively interact with gene products produced by expressing all of the genes in the group consisting of MYC, CDK4, CCNC, PTK2, BARD1, RAD 51, FANCG, PCNA, PRCC, TPR, EMS1, DSP, and HDGF.

11. The kit according to claim 9, wherein the at least one probe is substantially affixed to a solid support.

12. The kit according to claim 9, wherein the at least one probe is a polynucleotide that is complimentary to a unique portion of DNA present at least one of the genes selected from the group of genes consisting of; MYC, CDK4, CCNC, PTK2, BARD1, RAD 51, FANCG, PCNA, PRCC, TPR, EMS1, DSP, and HDGF and that binds to the unique portion of DNA under stringent conditions.

13. The kit according to claim 9, wherein the at least one probe includes a set of polynucleotides that is complimentary to the unique portions of all of the genes selected from the group of genes consisting of: MYC, CDK4, CCNC, PTK2, BARD1, RAD 51, FANCG, PCNA, PRCC, TPR, EMS1, DSP, and HDGF and that bind to the unique portions of DNA under stringent conditions.

14. The kit according to claim 9, further including a device used to measure the differential expression of the genes includes a chip wherein the chip is suitable for DNA annealation selection and ligation.

15. The kit according to claim 9, further including a set polynucleotide primers suited for use in quantitative real time PCR.

16. The kit according to claim 9, wherein the at least one probe is a protein that binds with high affinity to a protein produced by the expression of at least one of the genes selected from the group of genes consisting of: MYC, CDK4, CCNC, PTK2, BARD1, RAD 51, FANCG, PCNA, PRCC, TPR, EMS1, DSP, and HDGF.

17. The kit according to claim 16, wherein the protein is an antibody.

18. The kit according to claim 9, wherein the at least one probe is a set of proteins that bind with high affinity to the set of proteins produced by expressing all of the genes selected from the group consisting of: MYC, CDK4, CCNC, PTK2, BARD1, RAD 51, FANCG, PCNA, PRCC, TPR, EMS1, DSP, and HDGF.

19. The kit according to claim 18, wherein the proteins are antibodies.

20. The kit according to claim 9, further including instructions for correlating the level of gene expression measured for the genes selected from the group consisting of MYC, CDK4, CCNC, PTK2, BARD1, RAD 51, FANCG, PCNA, PRCC, TPR, EMS1, DSP, and HDGF, with a patient's likelihood of developing brain metastasis within three years of the patent's original cancer diagnosis.


SEQUENCE LISTING (39 sequences)

SEQ ID NO: 1 - DNA, 6 bp, Artificial Sequence
  Probe for CDK4 gene comprising a starting position at position 837 of accession number NM_0529
ggacac

SEQ ID NO: 2 - DNA, 6 bp, Artificial Sequence
  Probe for CDK4 gene comprising a starting position at position 377 of accession number NM_0529
catcag

SEQ ID NO: 3 - DNA, 6 bp, Artificial Sequence
  Probe for CDK4 gene comprising a starting position at position 781 of accession number NM_0529
ggagtg

SEQ ID NO: 4 - DNA, 7 bp, Artificial Sequence
  Probe for RAD51 gene comprising a starting position at position 1088 of accession number NM_0027
gatgttt

SEQ ID NO: 5 - DNA, 7 bp, Artificial Sequence
  Probe for RAD51 gene comprising a starting position at position 1387 of accession number NM_0027
tgttgtg

SEQ ID NO: 6 - DNA, 7 bp, Artificial Sequence
  Probe for RAD51 gene comprising a starting position at position 1542 of accession number NM_0087
ttgacat

SEQ ID NO: 7 - DNA, 7 bp, Artificial Sequence
  Probe for PRCC gene comprising a starting position at position 760 of accession number NM_00597
ctgcctt

SEQ ID NO: 8 - DNA, 7 bp, Artificial Sequence
  Probe for PRCC gene comprising a starting position at position 988 of accession number NM_00597
caaagca

SEQ ID NO: 9 - DNA, 7 bp, Artificial Sequence
  Probe for PRCC gene comprising a starting position at position 1223 of accession number NM_00597
gcctaag

SEQ ID NO: 10 - DNA, 7 bp, Artificial Sequence
  Probe for EMS1 gene comprising a starting position at position 755 of accession number NM_13856
gtttcgg

SEQ ID NO: 11 - DNA, 6 bp, Artificial Sequence
  Probe for EMS1 gene comprising a starting position at position 2014 of accession number NM_13856
cggcag

SEQ ID NO: 12 - DNA, 6 bp, Artificial Sequence
  Probe for EMS1 gene comprising a starting position at position 1739 of accession number NM_13856
ctgcgc

SEQ ID NO: 13 - DNA, 6 bp, Artificial Sequence
  Probe for PTK2 gene comprising a starting position at position 3719 of accession number NM_15383
tggcca

SEQ ID NO: 14 - DNA, 6 bp, Artificial Sequence
  Probe for PTK2 gene comprising a starting position at position 424 of accession number NM_15383
ggggca

SEQ ID NO: 15 - DNA, 7 bp, Artificial Sequence
  Probe for PTK2 gene comprising a starting position at position 3433 of accession number NM_15383
tctctag

SEQ ID NO: 16 - DNA, 6 bp, Artificial Sequence
  Probe for MYC gene comprising a starting position at position 1630 of accession number NM_00246
gaggac

SEQ ID NO: 17 - DNA, 6 bp, Artificial Sequence
  Probe for MYC gene comprising a starting position at position 1527 of accession number NM_00246
ggagtt

SEQ ID NO: 18 - DNA, 6 bp, Artificial Sequence
  Probe for MYC gene comprising a starting position at position 1344 of accession number NM_00246
tgccaa

SEQ ID NO: 19 - DNA, 6 bp, Artificial Sequence
  Probe for PCNA gene comprising a starting position at position 315 of accession number NM_18264
cagagc

SEQ ID NO: 20 - DNA, 6 bp, Artificial Sequence
  Probe for PCNA gene comprising a starting position at position 122 of accession number NM_18264
ctctga

SEQ ID NO: 21 - DNA, 6 bp, Artificial Sequence
  Probe for PCNA gene comprising a starting position at position 953 of accession number NM_18264
cttggc

SEQ ID NO: 22 - DNA, 6 bp, Artificial Sequence
  Probe for TPR gene comprising a starting position at position 3951 of accession number NM_00329
ggttga

SEQ ID NO: 23 - DNA, 6 bp, Artificial Sequence
  Probe for TPR gene comprising a starting position at position 5294 of accession number NM_00329
cagcca

SEQ ID NO: 24 - DNA, 6 bp, Artificial Sequence
  Probe for TPR gene comprising a starting position at position 3443 of accession number NM_00329
aagctc

SEQ ID NO: 25 - DNA, 6 bp, Artificial Sequence
  Probe for BARD1 gene comprising a starting position at position 1654 of accession number NM_00046
tctgcg

SEQ ID NO: 26 - DNA, 7 bp, Artificial Sequence
  Probe for BARD1 gene comprising a starting position at position 509 of accession number NM_00046
tggttta

SEQ ID NO: 27 - DNA, 7 bp, Artificial Sequence
  Probe for BARD1 gene comprising a starting position at position 2027 of accession number NM_00046
gaaattc

SEQ ID NO: 28 - DNA, 7 bp, Artificial Sequence
  Probe for DSP gene comprising a starting position at position 2858 of accession number NM_00441
agtcaca

SEQ ID NO: 29 - DNA, 7 bp, Artificial Sequence
  Probe for DSP gene comprising a starting position at position 3175 of accession number NM_00441
gcctcat

SEQ ID NO: 30 - DNA, 6 bp, Artificial Sequence
  Probe for DSP gene comprising a starting position at position 2592 of accession number NM_00441
ggcact

SEQ ID NO: 31 - DNA, 6 bp, Artificial Sequence
  Probe for HDGF gene comprising a starting position at position 1603 of accession number NM_00449
ggggct

SEQ ID NO: 32 - DNA, 6 bp, Artificial Sequence
  Probe for HDGF gene comprising a starting position at position 740 of accession number NM_00449
atgagc

SEQ ID NO: 33 - DNA, 6 bp, Artificial Sequence
  Probe for HDGF gene comprising a starting position at position 1258 of accession number NM_00449
gagtca

SEQ ID NO: 34 - DNA, 6 bp, Artificial Sequence
  Probe for FANCG gene comprising a starting position at position 2489 of accession number NM_00462
gtaggc

SEQ ID NO: 35 - DNA, 6 bp, Artificial Sequence
  Probe for FANCG gene comprising a starting position at position 1440 of accession number NM_00462
caaagc

SEQ ID NO: 36 - DNA, 6 bp, Artificial Sequence
  Probe for FANCG gene comprising a starting position at position 1004 of accession number NM_00462
gtggag

SEQ ID NO: 37 - DNA, 6 bp, Artificial Sequence
  Probe for CCNC gene comprising a starting position at position 891 of accession number NM_00519
gagagc

SEQ ID NO: 38 - DNA, 7 bp, Artificial Sequence
  Probe for CCNC gene comprising a starting position at position 567 of accession number NM_00519
tccagta

SEQ ID NO: 39 - DNA, 7 bp, Artificial Sequence
  Probe for CCNC gene comprising a starting position at position 708 of accession number NM_00519
ttgtaca